SEQUENCE LISTING (52 sequences)

SEQ ID NO: 1 - protein, 17 aa, Helicobacter pylori
  UNSURE (1): Unknown Amino Acid.
XPNGXYMXRXXXIXXXQ

SEQ ID NO: 2 - protein, 13 aa, Helicobacter pylori
  UNSURE (1): Unknown Amino Acid.
XKLXPQXGYVLMY

SEQ ID NO: 3 - protein, 11 aa, Helicobacter pylori
  UNSURE (1): Unknown Amino Acid.
XQDXFLXEGXS

SEQ ID NO: 4 - protein, 15 aa, Helicobacter pylori
  UNSURE (1): Unknown Amino Acid, probably Met.
XKKISRKEYVSMYGP

SEQ ID NO: 5 - protein, 15 aa, Helicobacter pylori
  UNSURE (1): Unknown Amino Acids.
XVNKPVKQTXAFGAP

SEQ ID NO: 6 - protein, 12 aa, Helicobacter pylori
  UNSURE (1): Unknown Amino Acid.
XFQVXFXIXAMN

SEQ ID NO: 7 - protein, 20 aa, Helicobacter pylori
  UNSURE (2)..(3): Unknown Amino Acids.
XXXGGFFTVGYQLGQVMQXV

SEQ ID NO: 8 - protein, 15 aa, Helicobacter pylori
  UNSURE (1)..(2): Unknown Amino Acids.
XXYEVHXXXINFXKV

SEQ ID NO: 9 - protein, 15 aa, Helicobacter pylori
  UNSURE (1)..(2): Unknown Amino Acids.
XXPGXFMTFGYELGQ

SEQ ID NO: 10 - protein, 14 aa, Helicobacter pylori
  UNSURE (1): Unknown Amino Acid.
XKEKFXRTKPXVXX

SEQ ID NO: 11 - protein, 10 aa, Helicobacter pylori
  UNSURE (1): Unknown Amino Acid.
XGHXQXHXAQ

SEQ ID NO: 12 - protein, 21 aa, Helicobacter pylori
  UNSURE (1): Unknown Amino Acid.
XEKNGAFVGISLEVGRADQKX

SEQ ID NO: 13 - protein, 20 aa, Helicobacter pylori
  urease A
MKLTPKELDKLMLHYAGELA

SEQ ID NO: 14 - protein, 14 aa, Helicobacter pylori
  UNSURE (1): Unknown Amino Acid.
XEFAQFVGVNYQXN

SEQ ID NO: 15 - protein, 25 aa, Helicobacter pylori
  UNSURE (1): Unknown Amino Acid.
XXSAAFVGVNYQVSMIQNQTKMVND

SEQ ID NO: 16 - protein, 15 aa, Helicobacter pylori
  UNSURE (1)..(3): Unknown Amino Acids.
XXXIXXXLYXLMLXR

SEQ ID NO: 17 - protein, 8 aa, Helicobacter pylori
  UNSURE (1): Unknown Amino Acid.
XQRMXQVG

SEQ ID NO: 18 - protein, 7 aa, Helicobacter pylori
  UNSURE (1): Unknown Amino Acid.
XLNIXFA

SEQ ID NO: 19 - protein, 15 aa, Helicobacter pylori
  UNSURE (1): Unknown Amino Acid.
XEQNXQNLQXXFFIX

SEQ ID NO: 20 - DNA, 2310 bp, Helicobacter pylori
  CDS (334)..(1851): protein
ttaaaaacatccaacaactctccttaaatttaaaaattcaaaaaataaaaatcaaaaaaaaaaaaaaacaaaatccgtcaatgcattgatataaaatagtataataattattattaaaaccagattaaaaataaaattttgtccttaatctttcttattttcattaattgttacgaatagaaatacttaaggggtttttttaattcttaaaaaaggattttttaaggaaattgaatcttgttagtctttgtataacaaattatgtaataatcaccacaagtgatcggcttagtgtcagattacgaagatttaagatcaattacaggaaaaaagatggttaataaagatgtgaaacaaaccactgcttttggcgctcccgtttgggatgacaacaatgtgattacggccggccctagaggtcctgttttattacaaagcacttggtttttggaaaagttagcggcgtttgacagagaaagaatccctgaaagggtggtgcatgctaaaggaagcggagcttatggcactttcactgtgactaaagacatcactaaatacactaaagcgaaaattttctctaaagtgggcaaaaaaaccgaatgcttcttcagattttccactgtggctggtgaaagaggcagtgcggatgcggtaagagaccctagaggttttgcgatgaagtattacactgaagaaggtaactgggatttagtagggaacaacacgcctgtcttctttatccgtgatgcgatcaaattccctgatttcatccacactcaaaaacgagatcctcaaaccaatttgcctaaccatgacatggtatgggatttttggagtaatgttcctgaaagcttataccaagtaacatgggttatgagcgatagagggattcctaaatctttccgccacatggatggttttggcagtcacactttcagtcttatcaacgctaaaggcgaacgcttttgggtgaaattccactttcacaccatgcaaggcgttaagcacttgactaacgaagaagccgcagaagtcagaaaatatgatcctgattccaatcaaagggatttattcaatgcgatcgctagaggggatttcccaaaatggaaattaagcgttcaagtgatgccagaagaagatgctaagaagtatcgattccatccgtttgatgtgactaaaatttggtacctccaagattatccgttgatggaagtgggcattgtagagttgaataaaaatcccgaaaactatttcgcagaagtggagcaagcggcattcagtccggctaatgtcgttcctggaattggctatagccctgataggatgttacaagggcgcttgttctcttatggggatacacaccgctaccgcttaggggttaattaccctcaaataccggttaataaaccaagatgcccgttccactcttctagcagagatggttacatgcaaaatgggtattacggctctttacaaaactatacgcctagctcattgcctggctataaagaagataagagcgcgagagatcctaaattcaacttagctcatattgagaaagagtttgaagtgtggaattgggattacagagctgatgatagcgattactacacccaaccaggtgattactaccgctcattgccagctgatgaaaaagaaaggttgcatgacactattggagagtctttggctcatgttacccataaggaaattgtggataaacaattggagcatttcaagaaagctgaccccaaatacgctgagggggttaaaaaagctcttgaaaaacaccaaaaaatgatgaaagacatgcatggaaaagacatgcaccacacaaaaaagaaaaagtaacccttttctttaagcgttcttattttttaggaacgctttgtctttcaaaatttaggtttttggatactcatcagtcctttggtggtgtgtcctattttttcattcattcaacgaatttaaaaattacaataaagagttatagttatgaaacgaagggattttattaaaacgactgctttaggcgctacaggtgctgttttaggagcacagattttgcaggcagaagaaagcaaagggagtgttgcaaaatataaaatagaagctcaatacagcattgattttgattctgcagaacacacttcgcttttcattcccatgccgagtgttgtagcgagcaatgtgcatttacaaggcaatcatgccagctataaaagcatgctcaattttggagtgccttatttgcaagtggattttttaaaaagcgctcaaaaaaagcaagtccatttgtcttatgagatc

SEQ ID NO: 21 - protein, 505 aa, Helicobacter pylori
  catalase from Helicobacter pylori
MVNKDVKQTTAFGAPVWDDNNVITAGPRGPVLLQSTWFLEKLAAFDRERIPERVVHAKGSGAYGTFTVTKDITKYTKAKIFSKVGKKTECFFRFSTVAGERGSADAVRDPRGFAMKYYTEEGNWDLVGNNTPVFFIRDAIKFPDFIHTQKRDPQTNLPNHDMVWDFWSNVPESLYQVTWVMSDRGIPKSFRHMDGFGSHTFSLINAKGERFWVKFHFHTMQGVKHLTNEEAAEVRKYDPDSNQRDLFNAIARGDFPKWKLSVQVMPEEDAKKYRFHPFDVTKIWYLQDYPLMEVGIVELNKNPENYFAEVEQAAFSPANVVPGIGYSPDRMLQGRLFSYGDTHRYRLGVNYPQIPVNKPRCPFHSSSRDGYMQNGYYGSLQNYTPSSLPGYKEDKSARDPKFNLAHIEKEFEVWNWDYRADDSDYYTQPGDYYRSLPADEKERLHDTIGESLAHVTHKEIVDKQLEHFKKADPKYAEGVKKALEKHQKMMKDMHGKDMHHTKKKK

SEQ ID NO: 22 - DNA, 2639 bp, Helicobacter pylori
  CDS (1)..(1086): protein HopY
gatctatcccaacaatacgctaatcagggtgtcattaagcctttggtggtggatgtggggaaagaacaaatcggtattactgatagcatgctcttggtggctcaaaacatcgttttagctttagggcaagtggatttgagcaaaatccaacaaaataaaaataatggtaacggacagctatacgaaaacatcatgaaagtcatgcttttaggtgcgggcgggactaatggagcgtataatggcgtgagtgtgggcgatattgccacaggcatgcaaaatttttcttcgcaaacgggcttgataggggctaattctacggttagcgagctcaacgctttgattaagagcgggatttctttagatcgtgagactttgaggttagggagttttattgaaaaaaatatttgtagcagtgcatcgtcttgttttactgggagtcagcttatctataagaaagggctagatagaaccataaacatcattaatgcggtattaggtcagtttgaatcttcggctagttctctttataagatttcttatatccctaacctcttttcgctcaaagattaccagtcagcgagcatgaacggctttggggctaagatgggttataaacaatttttcacccataagaaaaatattggcttaaggtattacgggtttttggattatggctatgcgaattttggcgatacgaatttaaaagtgggagcgaatcttgttacttatggggtaggaacggattttttatacaacgtgtatgaacgctctagaaggagggaaaggactacaatcggccttttctttggcgctcaaattgcagggcaaacttggagcactaatgtaacgaacttattgagcgggcaaaggcctgatgtcaagtctagttcgttccaattcttgtttgatttgggcgtgcgcaccaactttgcaaaaaccaatttcaacaagcacaggctagaccaagggatagaatttggggtgaaaatccctgttatcgctcataaatatttcgcaacccaaggctcaagcgcgagctatatgaggaattttagcttctatgtgggctattcagtcggtttttaaggaaggctcttgatgaaaaataccaatacaaaagagataaagaatacaagaatgaaaaaaggttatagtcaataccatgcgctcaaaaaagggcttttaaaaactgctctgctttttagccttcccttaagcatggcgttagctgaagacgatggcttttatatgggagtgggctatcaaatcggcggtgcgcaacaaaacatcaataacaaaggcagcaccctaaggaataatgtcattgatgatttccgccaagtgggcgtgggtatggcagggggtaacgggcttttagctttagcgacaaacacgaccatggacgctcttttagggataggcaaccaaattgtcaataccaatacaactgttggcaacaacaacgcagagttaacccagtttaaaaaaatactcccccaaattgagcgacgctttgagacgaataaaaacgcttatagcgttcaagccttgcaagtgtatttgagtaatgtgctttataacttggttaataatagtaataatggcagtaataatggagtcgttcctgaatatgtagggattataaaagttctctataattctcaaaatgaattcagtctcttagccacggagagtgtggcgcttttaaacgcgcttacaagggtgaatctggatagcaattcggtgtttttaaaagggctattagcccaaatgcagctttttaatgacacttcttcagcaaagctaggccagatcgcagaaaacttgaataagagtggtggtgcaggggccatgcttcaaaaggatgtgaaaaccatctcggatcgaatcgctacttaccaagagaatctaaaacaactaggagggatgctgaataattacgatgagccttacttgccccaatttgggccaggcaaaagctctcagcatggggttattaatggctttggcattcaagtgggctataagcaattttttgggagcaagaggaatataggcttacggtattacgctttctttgattatggctttacgcaattgggcagtcttaatagcgctgttaaagcgaacatctttacttatggcgctggcacggactttttatggaatatctttagaagggtttttagcgatcagtctttgaatgtgggggtgtttgggggcattcaaatagcgggtaacacttgggatagctctttaagaggccaaattgaaaactcgtttaaagaataccccactcccacgaatttccaatttttatttaatttgggcttaagggctcattttgccagcaccatgcaccgccggtttttgagctcgtctcaaagcattcagcatggtatggaatttggcgtgaaaatcccggctatcaatcaaaggtatttgaaagcgaatggggctgatgtggattacaggcgtttgtatgcgttctatatcaactacacgataggtttttaagctctttttagggcttataaagaggttctttacttttttttggtattctaacaagcttttaaaccatccaatctactttgttttaaggataatattttatggcagatgtcgttgtggggatc

SEQ ID NO: 23 - protein, 361 aa, Helicobacter pylori
DLSQQYANQGVIKPLVVDVGKEQIGITDSMLLVAQNIVLALGQVDLSKIQQNKNNGNGQLYENIMKVMLLGAGGTNGAYNGVSVGDIATGMQNFSSQTGLIGANSTVSELNALIKSGISLDRETLRLGSFIEKNICSSASSCFTGSQLIYKKGLDRTINIINAVLGQFESSASSLYKISYIPNLFSLKDYQSASMNGFGAKMGYKQFFTHKKNIGLRYYGFLDYGYANFGDTNLKVGANLVTYGVGTDFLYNVYERSRRRERTTIGLFFGAQIAGQTWSTNVTNLLSGQRPDVKSSSFQFLFDLGVRTNFAKTNFNKHRLDQGIEFGVKIPVIAHKYFATQGSSASYMRNFSFYVGYSVGF

SEQ ID NO: 24 - DNA, 2639 bp, Helicobacter pylori; identical to SEQ ID NO: 22
  CDS (1099)..(2517): signal peptide of protein

SEQ ID NO: 25 - protein, 472 aa, Helicobacter pylori
MKNTNTKEIKNTRMKKGYSQYHALKKGLLKTALLFSLPLSMALAEDDGFYMGVGYQIGGAQQNINNKGSTLRNNVIDDFRQVGVGMAGGNGLLALATNTTMDALLGIGNQIVNTNTTVGNNNAELTQFKKILPQIERRFETNKNAYSVQALQVYLSNVLYNLVNNSNNGSNNGVVPEYVGIIKVLYNSQNEFSLLATESVALLNALTRVNLDSNSVFLKGLLAQMQLFNDTSSAKLGQIAENLNKSGGAGAMLQKDVKTISDRIATYQENLKQLGGMLNNYDEPYLPQFGPGKSSQHGVINGFGIQVGYKQFFGSKRNIGLRYYAFFDYGFTQLGSLNSAVKANIFTYGAGTDFLWNIFRRVFSDQSLNVGVFGGIQIAGNTWDSSLRGQIENSFKEYPTPTNFQFLFNLGLRAHFASTMHRRFLSSSQSIQHGMEFGVKIPAINQRYLKANGADVDYRRLYAFYINYTIGF

SEQ ID NO: 26 - DNA, 1710 bp, Helicobacter pylori
  CDS (1)..(1179): mature peptide (without N-Terminus); protein HopZ of Helicobacter pylori
atcaaaaacgcccaagaaatcgtcgcacaagctcaaagccttaacaacccgcaaaacaatcaaaacgcgccgcaagatttcaatccttacacctctgctgatagggctttcgctcaaaacatgctcaatcacgcgcaagcgcaagccaagatgcttgaactagccaatcaaatcaaaaccaatcttagcgctatcccgcaacatttcaccaaagattacttggcagcttgccgcaatgggggtgggacattacctgatgcaggggttactaacaacacttggggagccggttgcgcctatgtggaagagaccataacggctttaaacaacagccttgtgcattttggcactcaagccgagcaaatcaagcaatctgagttgctggcgcgcacgatatttgattttaaaggcagccttaaggatttaaacagcacttataacagcatcaccacgaccgcttcaaacacgcccaattccccattccttaaaaatttgataagccaatccactaaccctaataaccccgggggcttacaggccgtttatcaagtcaaccaaagcgcttattcgcaattattaagcgccacgcaagaattagggcataaccctttcagacgctttggattaatcagctctcaaaccaacaatggtgccatgaatgggatcggtgtgcaaatagggtataaacaattttttggtgaaaagagaaaatggggggctaggtattacggcttttttgactataaccatgcttatatcaaatccagctttttcaactccgcctctgatgtgttcacttatggggtaggaacagatgtcctctataactttatcaacgataaagccaccaaaaacaataagatttcttttggggtgtttggggggattgctttagctggcacttcgtggctcaattctcaatacgtgaatttagcgaccttcaataatttctatagcgctaaaatgaatgtggcgaatttccaattcttattcaacttgggcttgagaatgaatctggctaaaaacaaaaagaaagcgagcgatcatgcggctcaacatggcgtggaactaggcgtgaagatccccacgatcaacacgaattactattctttgctaggcactcaactagaataccgcagactctatagcgtgtatttgaattatgtgtttgcgtattaaaagcttgcgttaaaccctttgtggaactccctttttaaggggtttcttttgaagccttttttttgaacctttttttgggggtcaagcgtaaaatccacccctatccctttaagaaaataaaataaaactttaagaactttaagaactttaagaaaatgcgttttacaacaaaataagatctaaaacaataaaacaaaaccccattttttaacaatgaaattttttaaacaaaaaagcattaaatcctaataaggtttgttagatcttgataaaaacaaagcttttttaaaaccccaaaaacaatactaaccaataaccaaaacgcatctattgtgatccttatagcataaaaccaagtttttatttaagcaaaagctgttatgccgttttaagagcgtttcgtttctatgaaaaccgcaatatttttcaattattcttgacaagcgttaaaaaaaattgtatcattatctttttgtgagacccgttagctcagttggtagagcaattcccttttaaggaatgggagcggcc

SEQ ID NO: 27 - protein, 392 aa, Helicobacter pylori
IKNAQEIVAQAQSLNNPQNNQNAPQDFNPYTSADRAFAQNMLNHAQAQAKMLELANQIKTNLSAIPQHFTKDYLAACRNGGGTLPDAGVTNNTWGAGCAYVEETITALNNSLVHFGTQAEQIKQSELLARTIFDFKGSLKDLNSTYNSITTTASNTPNSPFLKNLISQSTNPNNPGGLQAVYQVNQSAYSQLLSATQELGHNPFRRFGLISSQTNNGAMNGIGVQIGYKQFFGEKRKWGARYYGFFDYNHAYIKSSFFNSASDVFTYGVGTDVLYNFINDKATKNNKISFGVFGGIALAGTSWLNSQYVNLATFNNFYSAKMNVANFQFLFNLGLRMNLAKNKKKASDHAAQHGVELGVKIPTINTNYYSLLGTQLEYRRLYSVYLNYVFAY

SEQ ID NO: 28 - DNA, 2078 bp, Helicobacter pylori
  CDS (642)..(1388): signal peptide; 28 kD protein from Helicobacter pylori
gatccatcgctaaaagcattaattttgggctggtgtatggcatggggagtaagaaattgagcgaaactttaaacatttctttaaatgaggctaaaagctacatagaagcgtatttcaagcgattccctagcatcaaagattatttaaaccgcatgaaagaagagattttaaaaacttctaaagccttcactttgcttgggcgttatcgggtgtttgattttaccggcgcaaatgactatatcaagggcaattatttgcgagagggcgtgaatgcgatttttcaagggagtgccagcgatttattgaaattaggcatgctcaaagtgagcgagcgtttcaaaaataacccttcggtgaggctgcttttgcaagtgcatgacgaattgatttttgaaattgaagaaaaaaacgcattagagttgcagcaagaaatccaacgcattcttaataatgaagtgtatcctttgagggtgccactagaaacgagcgcgtttgtggcgaatcgttggaatgaactaaaaggttagttttttaattgagtttaagaaaaaatatattattatttctttataagtaatacttagctattttaagctaatataatatagagcgattatcaaaaaataaagggaaaagactgaatgttgaaaagaattatattattaggggctttgggtgttttagcgaacgctgaagagagcgcggcttttgtgggagtcaattaccaggtgagcatgatacaaaatcagactaaaatggtgaatgacaacggcttgcaaaagcctttgataaagttcccgccttatgcaggagcgggttttgaagtgggctataaacaattttttggcaagaaaaaatggtttggtgcgcgttattatgggttttttgactacgcgcacaaccgctttggcgtgatgaaaaagggtatcccggtgggcgagagcgggtttatttacaatagttttagttttggagggaacactttaatggaaagggattcctatcaagggcaatactatgtcaatttattcacttatggtgtggggctagatacgctgtggaattttgtgaataaagaaaacatggtttttggttttgtggtaggaatccaattagctggggatagttgggcaacgagcatcagtaaagagatcgccagctatgcaaaacaccacagcaattccagttatagcccggccaatttccagtttttatggaagtttggggtccgcacccatatcgctaaacacaatagcctagaattagggattaaagtgcctacgatcacgcaccggcttttctctcttaccaacgaaaagggatacaccttacaggctgatgtgcgccgagtttatgcgtttcaaatcagttacttgagggatttttaacccctttttagatacaatcgcacctaaaatcaatttaaaggtgtgaaatggtgaatttagaaaatttagactggaaaaatttaggctttagctacattaaaacggattttcgcttcatcgctacttataaaaacggctcttggtcgcatggcggattggtgagcgaaaatgtgctacaaatcagcgaaggctcgccggtcttgcactacgggcaggcttgttttgaaggcttgaaggcttaccgctctcaaaaggggaaggctttactttttcgccctttagaaaacgccaaacgcttgcaaacttcatgcgaaagactgctcatgcccaaagtgagcgaagagctgtttttaagggcatgcgctgaagtagtcaaagcgaatcaaaaatggctcgctccttataaaagcggggcgagtttgtatttgcgcccttttgtcataggcgtaggggataatttgggggtgaagccggctaatgaatacctttttatcgtgttttgcgcgcctgtgggggcgtattttaaggggggtatagaaaaaggaggggctaggtttatcactacgatttttgatagggccgcgcctaaaggcaccggaggggtgaaagtgggggggaattacgctgcaagcctgttagcccataaaatagccacagagcagggctatgatgattgcatttatttagatc

SEQ ID NO: 29 - protein, 248 aa, Helicobacter pylori
MLKRIILLGALGVLANAEESAAFVGVNYQVSMIQNQTKMVNDNGLQKPLIKFPPYAGAGFEVGYKQFFGKKKWFGARYYGFFDYAHNRFGVMKKGIPVGESGFIYNSFSFGGNTLMERDSYQGQYYVNLFTYGVGLDTLWNFVNKENMVFGFVVGIQLAGDSWATSISKEIASYAKHHSNSSYSPANFQFLWKFGVRTHIAKHNSLELGIKVPTITHRLFSLTNEKGYTLQADVRRVYAFQISYLRDF

SEQ ID NO: 30 - DNA, 3144 bp, Helicobacter pylori
  CDS (1149)..(2735): signal peptide; 50 kD membrane protein from Helicobacter pylori
gatcggcaggcaaatacacatctttattgcaacccattccttcgtatttttcaactttcaaggtccccaccaataattccttatgcttgcctttccaaagcgcggtcgtgtcgttggtggcatcatttttattcgcaaataccagatacatttggtattctatgggcttagttttaaggtgttgttggaatgaagaaagcagataatttgaatctttttgctttaattcttgggggttaagatacttaatgccctctttaggcacaaatttccatctcgcaggcaataatttttctttcttatctttaaacctgaacgcatgcacgctataatagggcgtgttagccacgcttgagctaatccctatcgttttggtgtaagcggcaaaattcttataagaggggacttcttcataaagctttttgattcttgcttcatccaccttgccatttttagggattctcatctcaaagaattgggcgaattcgttagggtttttggcaaaattgatttctgtattgagcatcaccattgtccagctagcgttttgattttctaattttaacgccattcccctaactttgcttttatcgtccattgccacgcctcctaaagaataccttacagatgcagggatttctttttcattgagtaatggcacatctaaatccttttttgcttgcgcattagggaggaacacgcctttagcacaaaaccccttagtgtggttgattttcattttaggctctttggcgttgagcttgtagaaaatatccgcaatctcttcagcgctcacttcatgggcttttaaaaaacccaagctaaaaaccaaacacaagctcaaaccaatttttttcattgtttcgctccttaattattatttttatataaaacaacgctttctattgtatcagtaaattccctattgagccttaaaaaagcctttttttcaatatattattaggtcttaaaaaattatcctatccatttgcatgctcatgagcaagcttttaaggataaacttgtgttttaaattttgtgatttttaagaaaaattagcttgattttaaactaattctatattcttttatgctacaattatttctacagagtaatttatctattctcaggtaaagtaaggaagaggaatgaaattaaagaaacgaaaagttgcggctacattgctaaagcgttttaccttaccactattgttcactacgggttcattaggggcggttacttatgaagtgcatggggattttatcaacttctccaaagtgggttttaaccattcgcccattaaccctgttaaaggtatctatcccacagagacttttgttaaccttacgggtaagctagagggttctgtgcatttaggtaggggatggaccgtgaatttaggcggtgttttgggcggacaggcttatgatggcactaagtatgataggtgggcgaaggattttacccccccaagctattgggataaaacttcttgcggcactgattctatgagcctttgtatgaatgctactaaaatgtggcaacaatcagggccaggtggtgtcattaaccctagaggtattggttgggaatatatgggtgagtggaacggcttgttccctaactactatccggctaacgcctacttgcctggtggctcaaggcgttatcaagtctataaagcaaatttgacctatgacagcgacagagtccatatggtaatggggcgttttgatattaccgagcaggagcaaatggattggatttaccaattgttccaagggttttatgggactttcaagctcactaagaatatgaaattcttgctctttagtggttggggtcgtggtatcgctgatggtcagtggttgttccctatctatcgtgaaaagccttggggggttcataaagcgggtattatttatcgccctacaaagaatttgatgatccacccttatgtgtatcttatcccaatggtaggcacattgcccggtgttaaagtagagtatgataccaatccggaatttagcggtaggggcattaggaataaaacgactttctatgcgttgtatgactatcgttggaataacgctgaatacggtcgttatgcgcccgctcgttataacacttgggatccgttcttggataatggtaagtggcgtggcttgcaaggtcctggcggtgcgacgcttcttttgcgccaccatatagatattaacaactattttgtggttggtggtgcttatctcaacattggtaaccctaacatgaacttaggtacttggggtaaccctgtggctcttgatggtatcgaacaatgggtcggtagtatctacagcttagggtttgcggggattgacaacattaccgatgctgatgcgttcaccgagtatgttaaaggtggaggcaagcatggtaagttcagttggagcgtttatcagcgcttcaccactgcaccaagggctttggaatatggtatcggtatgtatctagattatcagttcagcaagcatgttaaagcgggtctcaaactcgtatggttagagttccaaattcgtgcgggttacaaccctggaaccggtttccttgggccaaacggtcagccgcttaacttgaatactggtttgtttgagtcttcagcgttcgcgcaaggccctcaaaacatgggcggtatcgcaaaaagcatcactcaagacagaagccatttgatgacacacattagttatagtttctaagagagttctccccctatctcttagatatgccttttttgtatttttattttaatatctttgggagttagagttttggaaattaagaaatatttttcttactttctattttttttgcttttttctagtctctttttatccaaacttcaagcttataaattcaacatgagcattgttggaaaggtgagcagctataccaagtttggctttaacaaccaaagataccagccttctaaagacatttatcctacaggtagctacacttctttgctcggcgaattgaatttgagcatgggtttatacaagggtttgagggcggaagtgggggctatgatggcagcgctcccctatgactctaccgcctatcaaggcaacaatatccctaacggccagcccggctctaggaccgatc

SEQ ID NO: 31 - protein, 528 aa, Helicobacter pylori
MKLKKRKVAATLLKRFTLPLLFTTGSLGAVTYEVHGDFINFSKVGFNHSPINPVKGIYPTETFVNLTGKLEGSVHLGRGWTVNLGGVLGGQAYDGTKYDRWAKDFTPPSYWDKTSCGTDSMSLCMNATKMWQQSGPGGVINPRGIGWEYMGEWNGLFPNYYPANAYLPGGSRRYQVYKANLTYDSDRVHMVMGRFDITEQEQMDWIYQLFQGFYGTFKLTKNMKFLLFSGWGRGIADGQWLFPIYREKPWGVHKAGIIYRPTKNLMIHPYVYLIPMVGTLPGVKVEYDTNPEFSGRGIRNKTTFYALYDYRWNNAEYGRYAPARYNTWDPFLDNGKWRGLQGPGGATLLLRHHIDINNYFVVGGAYLNIGNPNMNLGTWGNPVALDGIEQWVGSIYSLGFAGIDNITDADAFTEYVKGGGKHGKFSWSVYQRFTTAPRALEYGIGMYLDYQFSKHVKAGLKLVWLEFQIRAGYNPGTGFLGPNGQPLNLNTGLFESSAFAQGPQNMGGIAKSITQDRSHLMTHISYSF

SEQ ID NO: 32 - DNA, 2825 bp, Helicobacter pylori
  CDS (891)..(2090): bp protein; 42 kD protein from Helicobacter pylori
gatcggaagcgagagtgataaaaggcattaggagagttagagacatcacttccacaaaagaagaaaaaaccgccatcagcacaagcaggaaaaaaaccattttttccttgaaagtgatgagcatatagatttgctttaaagaacgcaaaaagtattttaaagtagagattttatgtttttttttcgccataatttaagtgtccataaattcttttatatgtaataagcttgagctgtgttaagccaaattgagctagattatagctaaattttaaccatgctctgtgccatacgaataatttagctttctgccatcatttcttgacaagtcaagtataaaactgctataatcccaagtctttaatttgtttaatttgttgctggcttagctcagttggtagagcagctgccttgtaagcagcaggtcgggggttcaagtcccttagccagctccagttgaaatgttattgtgcaaagtttttggtgagatactcaagtggccaacgagggcagactgtaaatctgctgactatgtcttccgtggttcgaatccacgtctcaccaccattttgttttatagatgcgggaatagctcagttggctagagcatcagccttccaagctgagggtcgcgggttcgagtcccgtttcccgctccatttttaggataacattttagtttttgaggcgcctatatagctcagaggcagagcacttccttggtaaggaagaggtcggcggttcaattccgcttataggctccagtttataatctcttgaatggcgataagacaaaaatgtcttaaattttgtggtagcatttaggaatacttaggattttgtttagtataattctaaaatccatttcaaaaaattaaggagaaatacaaatggcaaaagaaaagtttaacagaactaagccgcatgttaatattggaaccattgggcatgtagaccatggtaaaacgactttgagtgcagcgatttcagcggtgctttctttgaaaggtcttgcagaaatgaaagactatgataatattgataacgcccctgaagaaaaagaaagagggatcactatcgctacttcccacattgaatatgagactgaaaacagacactatgcgcatgtggattgcccaggacacgctgactatgtaaaaaacatgatcaccggtgcggcacaaatggacggagcgattttggttgtttctgcagctgatggccctatgcctcaaaccagagagcatatcttattgtctcgtcaagtaggcgtgcctcacatcgttgttttcttaaacaaacaagacatggtagatgaccaagaattgttagagttggtagaaatggaagtgcgcgaattgttgagcgcgtatgaattccctggtgatgacactcctatcgtagcgggttcagctttaagagctttagaggaagcaaaggctggtaatgtgggtgaatggggtgaaaaagtgcttaagctcatggctgaagtggatgcctatatccctactccagaaagagacactgaaaaaactttcttgatgccggttgaagatgtgttctctattgcgggtagagggactgtggttacaggtaggattgaaagaggtgtggtgaaagtaggcgatgaagtggaaatcgttggtatcagagctacacaaaaaacgactgtaaccggtgtggaaatgtttagaaaagagctagaaaaaggtgaggccggcgataatgtgggcgtgcttttgagaggaactaaaaaagaagaagtagaacgcggtatggttctatgcaaaccaggttctatcactccgcacaagaaatttgagggagaaatttatgtcctttctaaagaagaaggcgggagacacactccattcttcaccaattaccgcccgcaattctatgtgcgcacgactgatgtgactggctctatcacccttcctgaaggcgtagaaatggttatgcctggcgataatgtgaaaatcactgtagagttgattagccctgttgcgttagagttgggaactaaatttgcgattcgtgaaggcggtaggaccgttggtgctggtgttgtgagcaatattattgaataatattagcaaaaagagttaccataaagggtcattatgaaagttaaaatagggttgaagtgttctgattgtgaagatatcaattacagcacaaccaagaacgctaaaactaacactgaaaaactggagcttaagaagttctgcccaagggaaaacaaacacactcttcataaagaaatcaaattgaagagctagttctttcttttgtgttgtgattgaaaaggaggggaggttaggtcagtagctccaatggtagagcgtcggtctccaaaaccggttgttgggggttcgagtccctcctggcctgccatctactaatttattctatcaaatttttgtttcaattggattgtttttgaattttttaattttagtttaagctattttggataaaattgaaaattcttttaatgtataaatattaagtttaagtgagggcgaaaagaaactatggataaatggctcatgcaatataaattagctagagaagagctttctaaagtgatatttcctattaaggagcagatacgcaacgcgcttgtttctgttttggtggtggtgagtgctatcacgctgtttttagctttgttggatttttctctgggggcttttatctctagtgttctataggttggtggctttaaataaggagaataatgatggattggtatgccatacaaacttattcagggagcgagcagtccgttaagaaagcgattgagaatctagcgaacgatc

SEQ ID NO: 33 - protein, 399 aa, Helicobacter pylori
MAKEKFNRTKPHVNIGTIGHVDHGKTTLSAAISAVLSLKGLAEMKDYDNIDNAPEEKERGITIATSHIEYETENRHYAHVDCPGHADYVKNMITGAAQMDGAILVVSAADGPMPQTREHILLSRQVGVPHIVVFLNKQDMVDDQELLELVEMEVRELLSAYEFPGDDTPIVAGSALRALEEAKAGNVGEWGEKVLKLMAEVDAYIPTPERDTEKTFLMPVEDVFSIAGRGTVVTGRIERGVVKVGDEVEIVGIRATQKTTVTGVEMFRKELEKGEAGDNVGVLLRGTKKEEVERGMVLCKPGSITPHKKFEGEIYVLSKEEGGRHTPFFTNYRPQFYVRTTDVTGSITLPEGVEMVMPGDNVKITVELISPVALELGTKFAIREGGRTVGAGVVSNIIE

SEQ ID NO: 34 - DNA, 2182 bp, Helicobacter pylori
  CDS (344)..(1507): signal peptide; 36/35/32 kD protein from Helicobacter protein
gatcgctctttgagtgattcctgtattcgctttattggcaaactcttcgccaaacattttcttcacattagggaaaattaccccatcaaaaaacaagtagccaataaaaataatggcgcacaataaatgaacaaccaacacataaggataaatcgcatccatttaaaatcctttattcatgggaaaattaaagagtttttaatctactataaaagggttttattgtcaagtatcccactattatgggaattttaggggtggtttttgtttgacttttaagattgcaattagctataataaaataattaaaaaagtaacacttaagcggagaccctagagagtgatgctcaattttatgacaaagaagaaaaatagaatgcaagattgcaaaatggttggtaaaaattttaatcgtaaggaatctgttttgatagctcaatctttagaaatttctaaaaaaggctcggtaattttaggcgctcttttgagttcgttatggctgacaaaccccttaaatgcccatgaaaagaatggcgcgtttgtggggattagcttggaagtgggtagggctgatcaaaagaccaacgcttatagaaacggcgagttgtttcaagtgccttttggcgatgtttcagccaatgatgatggcaaagtccctaacgggcagaccggtggctgtcagccagcttcagggacgccaggaacgccaggctatactaaagctaattgcgtggtcaattggacttctcgcaccatgcttagcaccaataaaaacattcctggccgtaaccagccgatgtatgggctaggtgtgatgacgggctataagcattttatcggtaaaaaaaggtggtttgggttgcgctattacggcttttttgattatgggcataccaatttctctaactccagggccgctaacgctatatcgcctttctatttgagcgatcaaaaagcggacatgtatacttatggttttggcacagacatgctttttaacattatagataagcctaaagccacggccgggttttttgtgggcgtgaattttgcgggtaacacttggaccaataatcgtgtggggtattttaaggacgggtatgtttatggcgtcaatacggatgctgacgcttacatgactaacgctgatggcacaatcacatgcggggacacgacgccggcgagttgtgatgtggggattaatcctaatagcgtctataccacaggaaaattgaacgctaaagtgaatcacacgattttccaatttttagtgaatgtgggcattagaactaatatttttgaacaccatggcattgagtttggtatcaaaatccccacgctccctaattactttttcaaaggctctactaccataagagcgaaaaaacaaggcccgctagagaatggccaaccaaccactatcaccggagcagaaaccaatttcagcttaacccaaaccttacgccgtcaatattctatgtatttgcgctatgtttatactttttgaatttggtagggtttttaggcagggcttatagcttatatatggatatatgaaagcttgatttgtcaagctttagggttgtcattgagttgcaataactctgtgctgttttctacttttttgataaaatcattaatggcataacagcgtatgttaatattgtctttgaaatgggcaaatcccgcatattctttggcgtcatcatggatatttggagctacaaaaacactaaatttttctctaatatcagtgctatttttaatcaattcttttaaatgtctggcaataggtatcatttccaaggtactttgacttctatctctaatcaagctcacttctatataactttgggcttttgtgtccatagctacaatatcaggtttgttaccgcttgctgtgtatacgggcaagccttcatcatcgcttttataattgggtatcacgcttaaattttcaaaatgttgtttcaagaaaatagcgcttaaaaattctaagcgtaaaggtttatcaatgagtcttaaaaaactatcttttgattcttgcttgttgcaagtaatgagtaattcttgcttgataaaatctttagtataagtggttgctagttcattcaatttgcttgttttaacgctctcatcagcgctgattggagtaacgctaacaagaaagctatccacgatc

SEQ ID NO: 35 - protein, 387 aa, Helicobacter pylori
MLNFMTKKKNRMQDCKMVGKNFNRKESVLIAQSLEISKKGSVILGALLSSLWLTNPLNAHEKNGAFVGISLEVGRADQKTNAYRNGELFQVPFGDVSANDDGKVPNGQTGGCQPASGTPGTPGYTKANCVVNWTSRTMLSTNKNIPGRNQPMYGLGVMTGYKHFIGKKRWFGLRYYGFFDYGHTNFSNSRAANAISPFYLSDQKADMYTYGFGTDMLFNIIDKPKATAGFFVGVNFAGNTWTNNRVGYFKDGYVYGVNTDADAYMTNADGTITCGDTTPASCDVGINPNSVYTTGKLNAKVNHTIFQFLVNVGIRTNIFEHHGIEFGIKIPTLPNYFFKGSTTIRAKKQGPLENGQPTTITGAETNFSLTQTLRRQYSMYLRYVYTF

SEQ ID NO: 36 - DNA, 480 bp, Helicobacter pylori
  CDS (1)..(480): bp mature protein; 17 kD protein from Helicobacter pylori
atggaacaaagccatcaaaacttgcaatctcaattttttatagagcatatcttacaaattctacctcaccgctatcccatgcttttagtggatagaattatagagttacaagccaataaaaaaattgtcgcttataagaatatcacttttaatgaagacgtgtttaacgggcatttccctaataagcccattttcccgggcgttttgatcgtagagggcatggcgcaaacgggagggtttttagccttcactagcttgtgggggtttgaccctgaaatcgccaaaacaaaaatcgtgtatttcatgacgattgataaggttaaattccgcatccctgtaaccccaggcgacagattagaataccatttagaagtcttaaagcataagggcatgatctggcaagtgggtggcacggctcaagtggatggcaaagtggtcgctgaagccgaattgaaagccatgattgcagagagagattaa

SEQ ID NO: 37 - protein, 159 aa, Helicobacter pylori
MEQSHQNLQSQFFIEHILQILPHRYPMLLVDRIIELQANKKIVAYKNITFNEDVFNGHFPNKPIFPGVLIVEGMAQTGGFLAFTSLWGFDPEIAKTKIVYFMTIDKVKFRIPVTPGDRLEYHLEVLKHKGMIWQVGGTAQVDGKVVAEAELKAMIAERD

SEQ ID NO: 38 - DNA, 42 bp, Artificial Sequence
  misc_feature (3): N is inosine
gtnaayaaagatgtnaaacaaacytgtgcntttggcgcncct

SEQ ID NO: 39 - DNA, 45 bp, Artificial Sequence
  Description of Artificial Sequence  probes
ggcggcttttttacygtgggctatcaattrggccaagtgatgcaa

SEQ ID NO: 40 - DNA, 44 bp, Artificial Sequence
  Description of Artificial Sequence  probes
gtgacytatgaagtgcatggcgattttatyaaytttagcaaagt

SEQ ID NO: 41 - DNA, 27 bp, Artificial Sequence
  Description of Artificial Sequence  probes
aaagaaaaatttaacagaacyaaacct

SEQ ID NO: 42 - DNA, 57 bp, Artificial Sequence
  misc_feature (12): N is inosine
gaaaaaaatggngcntttgtgggcatyagcttngaggttggnagagctgatcaaaaa

SEQ ID NO: 43 - DNA, 72 bp, Artificial Sequence
  Description of Artificial Sequence  probes
tggagcgctgcttttgtgggcgtgaattatcaagtgagcatgatycaaaatcaaacyaaaatggtgaatgat

SEQ ID NO: 44 - protein, 24 aa, Helicobacter pylori
EDDGGFFTVGYQLGQVMQDVQNPG

SEQ ID NO: 45 - protein, 14 aa, Artificial Sequence
  UNSURE (9): Unknown Amino Acid
VNKDVKQTXAFGAP

SEQ ID NO: 46 - protein, 15 aa, Artificial Sequence
  Description of Artificial Sequence  probes
GGFFTVGYQLGQVMQ

SEQ ID NO: 47 - protein, 15 aa, Artificial Sequence
  Description of Artificial Sequence  probes
VTYEVHGDFINFSKV

SEQ ID NO: 48 - protein, 9 aa, Artificial Sequence
  Description of Artificial Sequence  probes
KEKFNRTKP

SEQ ID NO: 49 - protein, 19 aa, Artificial Sequence
  Description of Artificial Sequence  probes
EKNGAFVGISLEVGRADQK

SEQ ID NO: 50 - protein, 24 aa, Artificial Sequence
  Description of Artificial Sequence  probes
WSAAFVGVNYQVSMIQNQTKMVND

SEQ ID NO: 51 - protein, 23 aa, Artificial Sequence
  Description of Artificial Sequence probes
RDPKFNLAHIEKEFEVWNWDYRA

SEQ ID NO: 52 - protein, 22 aa, Artificial Sequence
  Description of Artificial Sequence probes
EKHQKMMKDMHGKDMHHTKKKK

(19) United States
(12) Patent Application Publication
(10) Pub. No.: US 2005/0063987 A1
(43) Pub. Date: Mar. 24, 2005

(54) Proteins, in particular membrane proteins, of Helicobacter pylori, their preparation and use

(75) Inventors: Bernhard Knapp, Marburg/Lahn (DE); Erika Hundt, Marburg/Lahn (DE); Karl-Heinz Schmidt, Marburg (DE)

Correspondence Address: Carol P. Einaudi, Esquire;Finnegan, Henderson, Farabow, Garrett & Dunner, 1300 I Street, NW, Washington, DC, 20005-3315, US

(73) Assignee: CHIRON BEHRING GmbH & CO

(21) Appl. No.: 10/662,126

(22) Filed: Sep. 12, 2003

Related U.S. Application Data
(63) Continuation of application No. 09/230,158, filed on Jan. 19, 1999, now abandoned.

(30) Foreign Application Priority Data
Jul. 26, 1996 (DE) 19630390.7

(51) Int. Cl. A61K 39/02; C07K 14/195

(52) U.S. Cl. 424/190.1; 530/350

(57) ABSTRACT

The present invention relates to novel proteins, in particular membrane proteins or proteins which are firmly associated with the membrane, which are derived from Helicobacter pylori (H. pylori) and which contain the peptide sequence SEQ ID NO:37 according to Tables 1a-1c, or to parts or homologues thereof having a minimum length of ten amino acids, and to their preparation and use as pharmaceutical compositions, in particular as vaccines, or as a diagnostic agent. Based on these data, the gene coding for this protein was also isolated as shown in SEQ ID NO: 36.

### TECHNICAL FIELD OF THE INVENTION

The present invention relates to novel proteins, in particular membrane proteins or proteins which are firmly associated with the membrane, which are derived from Helicobacter pylori (H. pylori) and which contain one of the peptide sequences selected from SEQ ID NO: 1, 2, 3, 6, 10, 11, 12, 14, 15, 16, 17, 18 or 19 according to Tables 1a-1c, or to parts or homologues thereof having a minimum length of five amino acids, and to their preparation and use as pharmaceutical compositions, in particular as vaccines, or as a diagnostic agent. Based on these data, genes coding for these and related proteins were also isolated as shown in SEQ ID NOS: 20, 21, 22, 23, 24, 25, 26 and 27.

### BACKGROUND OF THE INVENTION

Helicobacter pylori is a Gram-negative, microaerophilic, spiral bacterium which colonizes the mucosa of the human stomach. The bacterium is the cause of chronic active gastritis and of peptic ulcer, in particular duodenal ulcer, and plays a role in the development of carcinomas of the stomach; consequently, Helicobacter pylori is an important human pathogen.

Its helical shape and motility, due to from four to six flagellae, enables the bacterium to migrate through the gastric mucus in order to reach the boundary layer, which is virtually at neutral pH, between the mucus and the mucosa. Ammonium ions, which are produced during the enzymic cleavage of urea by bacterial urease, protect the pathogen from the aggressive gastric acid. The bacterium adheres to the endothelial cells of the stomach using specific adhesins.

A consequence of chronic colonization of the mucosa can be an inflammatory granulocytic, and subsequently monocytic, infiltration of the epithelium which in turn, by way of inflammation mediators, contributes to the tissue destruction. Infection stimulates both a local and a systemic humoral immune response, without these responses being able to eliminate the pathogen effectively. Immunization is the conventional way of preventing infectious diseases. It is therefore important to examine this option with regard to controlling an H. pylori infection.

The development of a vaccine involves identifying factors which are crucial for virulence or structures which are accessible to the human immune system for the purpose of eliminating a pathogen. It is to be assumed that antigens of this nature are present in the outer membrane of the bacterium. Thus, adhesins of 19,600 Da (P. Doig et al., 1992, J. of Bacteriology 174, 2539-2547), 20,000 Da (D. G. Evans et al., 1993, J. of Bacteriology 175, 674-683) and 63,000 Da (C. Lingwood et al., 1993, Infection and Immunity 61, 2474-2478) are located in the outer membrane, which adhesins are candidates for an experimental vaccine which has the aim of inducing antibodies which prevent adhesion of the bacterium to the mucosal surface.

In addition, the outer membrane possesses porins of 30,000 Da (M. A. Tufano et al., 1994, Infection and Immunity 62, 1392-1399), 48,000 Da, 49,000 Da, 50,000 Da, 67,000 Da (M. M. Exper et al., 1995, Infection and Immunity 63, 1567-1572) and 31,000 Da (P. Doig et al., 1995, J. of Bacteriology 177, 5447-5452) molecular weight, and also iron-regulated out r membrane proteins of 77,000 Da, 50,000 Da and 48,000 Da (D. J. Worst et al., 1995, Infection and Immunity 63, 4161-4165) molecular weight, erythrocyte-binding antigens of 59,000 Da and 25,000 Da (J. Huang et al., 1992, J. of Gen. Microbiol. 138, 1503-1513) molecular weight and proteins for binding laminin, collagen I and IV, fibronectin and vitronectin (I. Kondo et al., 1993, European J. Gastroenterol. Hepatol. 5, 63-67). In addition, proteins of 19,000 Da (E. B. Drouet et al., 1991, J. of Clinical Microbiology 29, 1620-1624), 50,000 Da (M. M. Exner et al., 1995, Infection and Immunity 63, 1567-1572) and 30,000 Da (J. Bölin et al., 1995, J. of Clinical Microbiology 33, 381-384) molecular weight, and also a 20,000 Da lipoprotein (M. Kostrzynska et al., 1994, J. of Bacteriology 176, 5938-5948) and strain-specific, surface-located antigens of 51,000 Da, 60,000 Da and 80,000 Da (P. Doig and T. J. Trust, 1994, Infection and Immunity 62, 4526-4533) have been described. The genes for the proteins of 20,000 Da (HpaA) (Evans et al.) and 20,000 Da (lpp20) (M. Kostrzynska et al.) molecular weight have now been isolated. N-terminal protein sequence data have been disclosed for the adhesins of 19,600 Da (P. Doig et al., 1992) and 63,000 Da (C. Lingwood et al.) molecular weight, for the porins of 48,000 Da, 49,000 Da, 50,000 Da, 67,000 Da (M. M. Exner et al.), 30,000 Da (M. A. Tufano, 1994) and 31,000 Da (P. Doig et al., 1995) molecular weight and for the 50,000 Da protein (M. M. Exner et al., 1995).

### SUMMARY OF THE INVENTION

According to a first aspect of the present invention there is provided a protein from Helicobacter pylori (H. pylori) containing one of the peptide sequences selected from SEQ ID NO: 1, 2, 3, 6, 10, 11, 12, 14, 15, 16, 17, 18 and 19 according to Tables 1a-1c, or parts or homologues there of having a minimum length of five amino acids. Preferably the peptide sequences of the protein are N-terminal sequences.

The protein according to the first aspect of the present invention preferably contains a peptide sequence having the SEQ ID NO: 1 according to Table la and has a molecular weight of approx. 250 kD, or preferably contains a peptide sequence having the SEQ ID NO: 2 according to Table 1a and has a molecular weight of approx. 110 kD, or preferably contains a peptide sequence having the SEQ ID NO: 3 according to Table 1a and has a molecular weight of approx. 100 kD, or preferably contains a peptide sequence having the SEQ ID NO: 6 according to Table 1a and has a molecular weight of approx. 60 kD, or preferably contains a peptide sequence having the SEQ ID NO: 10 according to Table 1b and has a molecular weight of approx. 42 kD, or preferably contains a peptide sequence having the SEQ ID NO: 11 according to Table 1b and has a molecular weight of approx. 42 kD, or preferably contains a peptide sequence having the SEQ ID NO: 12 according to Table 1b and has a molecular weight of from approx. 32 to approx. 36 kD, or preferably contains a peptide sequence having the SEQ ID NO: 14 according to Table 1c and has a molecular weight of approx. 30 kD, or preferably contains a peptide sequence having the SEQ ID NO: 15 according to Table 1c and has a molecular weight of approx. 28 kD, or preferably contains a peptide sequence having the SEQ ID NO: 16 according to Table 1c and has a molecular weight of approx. 28 kD, or preferably contains a peptide sequence having the SEQ ID NO: 17 according to Table 1c and has a molecular weight of approx. 25 kD, or preferably contains a peptide sequence having the SEQ ID NO: 18 according to Table 1c and has a molecular weight of approx. 25 kD, or preferably contains a peptide sequence having the SEQ ID NO: 19 according to Table 1c and has a molecular weight of approx. 17 kD.

The protein according to the first aspect of the present invention is preferably a membrane protein or a protein which is firmly associated with the membrane. More preferably said protein is an integral membrane protein, in particular a Sarkosyle-insoluble integral membrane protein.

In a second aspect of the invention there are provided proteins according to the first aspect of the present invention, which can be obtained in accordance with the following procedural steps:
(a) isolating the proteins by means of differential solubilization;
(b) separating the proteins, which have been isolated in accordance with step (a), by means of gel electrophoretic methods; and
(c) isolating the proteins, which have been separated in accordance with step (b).

Preferably the proteins according to the second aspect of the present invention can be obtained by means of differential solubilization using Sarkosyle. The proteins can also be obtained by means of separation by one or more SDS polyacrylamide gel electrophoreses, preferably by means of several SDS polyacrylamide gel electrophoreses having different polyacrylamide contents, more preferably wherein the polyacrylamide content of said gel electrophoreses is approximately 8%, 10% or 16%.

In a third aspect of the present invention there is provided a peptide having the amino acid sequence according to SEQ ID NO: 1, 2, 3, 6, 10, 11, 12, 14, 15, 16, 17, 18 or 19 according to Tables 1a-1c, or parts or homologues thereof having a minimum length of five amino acids.

In a fourth aspect of the present invention there is provided an antibody against on or more proteins according to the first or second aspects of the present invention and/or against one or more peptides according to the third aspect of the present invention.

In a fifth aspect of the present invention there is provided a polynucleotide encoding one or more proteins according to the first or second aspects of the present invention or one or more peptides according to the third aspect of the present invention.

In a sixth aspect of the present invention there is provided a process for preparing the proteins according to the first or second aspects of the present invention, characterized in that the following procedural steps are carried out:
(a) isolating the proteins, by means of differential solubilization;
(b) separating the proteins, which have been isolated in accordance with step (a), by means of gel electrophoretic methods; and
(c) isolating the proteins, which have been separated in accordance with step (b).

Preferably the process is characterized in that the proteins are isolated in accordance with step (a) using Sarkosyle.

In a seventh aspect of the present invention there is provided a process for preparing the peptides according to the third aspect of the present invention, characterized in that a chemical peptide synthesis is carried out.

In an eighth aspect of the present invention there is provided a process for preparing the proteins according to the first or second aspects of the present invention or the peptides according to the third aspect of the present invention, characterized in that a polynucleotide according to the fifth aspect of the present invention is expressed.

In a ninth aspect of the present invention there is provided the use of one or more proteins according to the first or second aspects of the present invention, one or more peptides according to the third aspect of the present invention, one or more antibodies according to the fourth aspect of the present invention or one or more polynucleotides according to the fifth aspect of the present invention for preparing a pharmaceutical composition or a diagnostic agent.

In a tenth aspect of the present invention there is provided a pharmaceutical composition comprising one or more proteins according to the first or second aspects of the present invention and/or one or more peptides according to the third aspect of the present invention or one or more antibodies according to the fourth aspect of the present invention or one or more polynucleotides according to the fifth aspect of the present invention or their expression products. Preferably said pharmaceutical composition is used as a vaccine.

In an eleventh aspect of the present invention there is provided a diagnostic agent comprising one or more proteins according to the first or second aspects of the present invention and/or one or more peptides according to the third aspect of the present invention or one or more antibodies according to the fourth aspect of the present invention or one or more polynucleotides according to the fifth aspect of the present invention or their expression products.

In a twelfth aspect of the present invention there is provided a protein from H. pylori containing one of the peptide sequences deduced from SEQ ID NO: 21, 22, 23, 24, 25, 26 and 27, or parts or homologues thereof having a minimum length of five amino acids.

In a thirteenth aspect of the present invention there is provided a peptide having the amino acid sequence deduced from SEQ ID NO: 21, 22, 23, 24; 25, 26 or 27, or parts or homologues thereof having a minimum length of five amino-acids.

In a fourteenth aspect of the present invention there is provided a peptide selected from the C-terminal region of the peptide sequence of SEQ ID NO: 20 or homologue thereof. Preferably said peptide is selected from RDPKFNLAHIHEKEFEVWNWDYRA and EKHQKMMKDMHGKDMHHTKKKK, or parts or homologues thereof.

In a fifteenth aspect of the present invention there is provided an antibody against one or more proteins according to the twelfth aspect of the present invention and/or against one or more peptides according to the thirteenth or fourteenth aspects of the present invention.

In a sixteenth aspect of the present invention there is provided a polynucleotide encoding one or more proteins according to the twelfth aspect of the present invention or one or more peptides according to the thirteenth or fourteenth aspects of the present invention.

In a seventeenth aspect of the present invention there is provided a host cell transformed with the polynucleotide according to the fifth or sixteenth aspects of the present invention.

In an eightenth aspect of the present invention there is provided an expression product expressed from th host cell according to the seventeenth aspect of the present invention.

In a nineteenth aspect of the present invention there is provided a pharmaceutical composition comprising one or more proteins according to the twelfth aspect of the present invention and/or one or more peptides according to the thirteenth or fourteenth aspects of the present invention or one or more antibodies according to the fifteenth aspect of the present invention or one or more polynucleotides according to the sixteenth aspect of the present invention or their expression products. Preferably said pharmaceutical composition is used as a vaccine. More preferably, when the pharmaceutical composition comprises a nucleotide sequence, said pharmaceutical composition is used as a DNA vaccine.

In a twentieth aspect of the present invention there is provided a diagnostic agent comprising one or more proteins according to the twelfth aspect of the present invention and/or one or more peptides according to the thirteenth or fourteenth aspects of the present invention or one or more antibodies according to the fifteenth aspect of the present invention or one or more polynucleotides according to the sixteenth aspect of the present invention or their expression products.

In a twenty-first aspect of the present invention there is provided the use of one or more proteins according to the twelfth aspect of the present invention or one or more peptides according to the thirteenth or fourteenth aspects of the present invention or one or mor antibodies according to the fifteenth aspect of the present invention or one or more polynucleotides according to the sixteenth aspect of the present invention or their expression products for preparing a pharmaceutical composition or a diagnostic agent.

### DETAILED DESCRIPTION OF THE INVENTION AND BEST MODE

The present application describes the isolation and determination of, in all, 19 proteins, in particular membrane proteins or proteins which are firmly associated with the membrane, especially integral membrane proteins, which proteins are in a molecular weight range of from 17 kD to approx. 250 kD (Tables 1a-1c). The term membrane protein is generally understood to mean integral and peripheral membrane proteins and transmembrane proteins. Integral membrane proteins are proteins which are partially or entirely inserted into the cytoplasmic membrane. By contrast, peripheral membrane proteins only adhere to the surface of the membrane. Transmembrane proteins pass completely through the membrane (see, for example, B. Alberts et al. (eds), Membrane Proteins in “Molecular Biology of the Cell”, 2nd ed., Garland Publishing, Inc., New York & London, 284-287, 1989). Two sequences were identified in one band in seven cases (SEQ ID NO: 2 and 3, 5 and 6, 7 and 8, 10 and 11, 13 and 14, 15 and 16, and 17 and 18), while it was only possible to identify one sequence in one band in a further five cases (SEQ ID NO: 1, 4, 9, 12 and 19). Six N-terminal sequences from the 19 peptide sequences identified had already been described in earlier studies; these were the sequences for urease A and urease B (B. E. Dunn et al., 1990, J. Biolog. Chem. 265, 9464-9469), for the exoenzyme S-like protein (C. Lingwood et al.), for the 50 kD membrane protein and for the porins hop B and hop C (M. M. Exner et al.). The only genes for these antigens which have so far been isolated are those for urease A and urease B (A. Labigne et al., 1991, J. Bacteriol. 173, 1920-1931). It was not possible to find the N-terminal sequences, which have already been described, of the membrane proteins of 19,600 Da (P. Doig et al., 1992), 48,000 Da, 67,000 Da (M. M. Exner et al., 1995) and 31,000 Da (P. Doig et al., 1995) molecular weight among the 19 sequences which are described in accordance with the invention. Thus, the protein which is described by SEQ ID NO: 14 cannot be attributed, either, to the protein having the molecular weight of 31,000 Da (P. Doig et al., 1995). The remaining 13 amino terminal protein sequences of the 19 amino terminal protein sequences according to Tables 1a-1c have not been described. It is to be assumed that these sequences can be attributed to Helicobacter pylori proteins which have not previously been identified.

It was surprising, therefore, that it was possible to demonstrate a large number of additional, novel H. pylori proteins in a Sarkosyle-insoluble fraction. The proteins are very probably integral proteins of the outer membrane or proteins which are firmly associated with the membrane. They are therefore particularly suitable for use as candidates for developing a vaccine or a diagnostic agent.

The invention describes proteins, in particular membrane proteins or proteins which are firmly associated with the membrane, especially integral membrane proteins, in particular Sarkosyle-insoluble integral membrane proteins of H. pylori, which contain one of the peptide sequences selected from SEQ ID NO: 1, 2, 3, 6, 10, 11, 12, 14, 15, 17, 18 or 19 according to Tables 1a-1c, or to parts or homologues thereof having a minimum length of five, preferably six amino acids, with these peptide sequences preferably constituting N-terminal sequences of the said proteins. The novel peptides are particularly preferred which exhibit at least ten consecutive amino acids selected from th sequences having the SEQ ID NO: 1, 2, 3, 6, 10, 11, 12, 14, 15, 16 and 19. In addition, those said parts are in particular preferred which contain an uninterrupted sequence of unambiguously specified amino acids.

The term “part” in the context of “part(s) of a sequence” in the present invention is defined herein as meaning a sequence of amino acids which can form a T-cell or B-cell epitope. Such an amino acid sequence is usually of a minimum of approximately four to eight amino acids.

The term “homologue(s)” in the context of the present invention is defined herein as meaning the same protein or peptide of a different strain of H. pylori but exhibiting the same function. Thus, although the actual amino acid sequences may not be identical between homologous proteins or peptides from different strains of H. pylori, the differences between the amino acid sequences merely represent strain-specific differences; the function of the homologues is identical.

In a particular embodiment, the protein containing a peptide sequence having the SEQ ID NO: 1 according to Table 1a has a molecular weight of approx. 250 kD, the protein containing a peptide sequence having the SEQ ID NO: 2 according to Table 1a has a molecular weight of approx. 110 kD, the protein containing a peptide sequence having the SEQ ID NO: 3 according to Table 1a has a molecular weight of approx. 100 kD, the protein containing a peptide sequence having the SEQ ID NO: 6 according to Table 1a has a molecular weight of approx. 60 kD, the protein containing a peptide sequence having the SEQ ID NO: 10 according to Table 1b has a molecular weight of approx. 42 kD, the protein containing a peptide sequence having the SEQ ID NO: 11 according to Table 1b has a molecular weight of approx. 42 kD, the protein containing a peptide sequence having the SEQ ID NO: 12 according to Table 1b has a molecular weight of from approx. 32 to approx. 36 kD, the protein containing a peptide sequence having th SEQ ID NO: 14 according to Tabl 1c has a molecular weight of approx. 30 kD, the protein containing a peptide sequence having the SEQ ID NO: 15 according to Table 1c has a molecular weight of approx. 28 kD, the protein containing a peptide sequence having the SEQ ID NO: 16 according to Table 1c has a molecular weight of approx. 28 kD, the protein containing a peptide sequence having the SEQ ID NO: 17 according to Table 1c has a molecular weight of approx. 25 kD, the protein containing a peptide sequence having the SEQ ID NO: 18 according to Table 1c has a molecular weight of approx. 25 kD, and the protein containing a peptide sequence having the SEQ ID NO: 19 according to Table 1c has a molecular weight of approx. 17 kD.

The generally available H. pylori strain No. ATCC 43504 is used, for example, as the starting material when isolating the proteins, with it being possible, in particular, to carry out the following procedural steps:
(a) isolating the proteins by means of differential solubilization, in particular using Sarkosyle (an N-lauroylsarcosine) in accordance with the method of Blaser et al. (1983, Infect. Immun. 42, 276-284),
(b) separating the proteins, which have been isolated in accordance with step (a), by means of gel electrophoretic methods, preferably by means of SDS polyacrylamide gel electrophoresis, with use being made, in particular, of polyacrylamide gels having differing polyacrylamide contents, in particular containing approx. 8, 10 or 16% polyacrylamide, and
(c) isolating the proteins, which have been separated in accordance with step (b), by means of known methods, for example by elution or by isolation on a membrane.

For the purpose of isolating and characterizing the proteins according to the present invention, the proteins were first of all obtained using the method of Blaser et al. (see above). The bacteria, which had been disrupted in a glass bead homogenizer, were freed of intact bacteria by centrifugation at 5000 g; the supernatant was then centrifuged at 100,000 g. The pellet was dissolved in Sarkosyl, and the Sarkosyle-insoluble fraction, which contains the integral membrane proteins in particular, was centrifuged off. The pellet was resuspended in distilled water and fractionated by SDS polyacrylamide gel electrophoresis (PAGE). In this connection, it was found that SDS-PAGE, in contrast to HPLC, was a very effective method for separating Sarkosyle-insoluble proteins. For this, the gels were pretreated with methionine in order to prevent oxidation of the methionine residues. After the run, the proteins were transferred from the SDS gel to a PVDF membrane (Immobilon Pe, from Millipore), with 0.005% SDS being added to the cathode buffer in order to complete the transfer of the very insoluble proteins. For sequence analysis, the protein bands from four tracks, in each case, were cut out of the PVDF membrane and Edman amino acid degradation was carried out in a 477A fluid-phase sequencer (Applied Biosystems, Inc. (ABI)) to determine the amino acid sequence. While it is possible further to fractionate the proteins which run in one band, for example by means of isoelectric focusing or two-dimensional gel electrophoresis, this is not necessary for an unambiguous sequence analysis since the sequences can be assigned unambiguously on the basis of the different protein contents of the proteins which run in one band.

The amino acids which are labelled Xaa in the sequence listing can be explained as follows:

The non-identifiable amino acids can be caused by interference due to impurities in the first sequencing step, a non-analysable amino acid, such as Cys or Trp, a modifiable amino acid which is missing in the elution programme, or an amino acid, such as Ser or Thr, which is difficult to determine, basically due to low sequence yields. Differ nt bands can also contain two proteins of very similar molecular weights in different quantities. This then results in two sequences which then also have to be assigned unambiguously on account of the different frequency of the individual amino acids.

The present invention also describes the peptides which are designated by the sequences according to SEQ ID NO: 1, 2, 3, 6, 10, 11, 12, 14, 16, 17, 18 or 19 according to Tables 1a-1c, or to parts or homologues thereof having a minimum length of five amino acids, in particular of six amino acids, which can be prepared, for example, by well-known chemical peptide synthesis (Barani, G. & Merrifield, R. B. in “The Peptides: Analysis, Synthesis and Biology” (Gross E., ed.), Vol. 2, Academic Press, 1980, Johannes Meyenhofer Verlag; Bodanszky, M. & Bodanszky, A. “The practice of peptide synthesis”, Springer Verlag, 1984). The novel peptides are particularly preferred which possess at least ten consecutive amino acids selected from the sequences having the SEQ ID NO: 1, 2, 3, 6, 10, 11, 12, 14, 15, 16 and 19. Furthermore, those said peptides are, in particular, preferred which contain an uninterrupted sequence of unambiguously determined amino acids, as is the case with the sequences from SEQ ID NO: 12, 14 and 15.

The present application also describes antibodies which can also be prepared by methods which are well known to the skilled person (see, for example, B. A. Diamond et al. (1981), The New England Journal of Medicine, 1344-1349) and which are directed against one or more of the novel proteins or peptides.

The skilled person is also familiar, from J. Sambrook et al. (1989, “Molecular Cloning, A Laboratory Manual”, 2nd edn., Cold Spring Harbor Laboratory, Cold Spring Harbor, N.Y.), with methods for preparing polynucleotides which encode the novel proteins or peptides. In particular, the skilled person knows, on the basis of the genetic code, the nucleotide sequences which encode the peptides according to the sequence listing. In particular, the nucleotide sequences are preferred which occur most frequently in accordance with the rules for the frequency of us of the different codons in Helicobacter pylori. These nucleotide sequences can be prepared, for example, by means of chemical polynucleotide synthesis (see, for example, E. Uhlmann & A. Peyman (1990), Chemical Reviews, 543-584, Vol. 90, No. 4).

For example, oligodeoxynucleotides which have been prepared in accordance with these rules can be employed for screening Helicobacter pylori gene libraries using known methods (J. Sambrook et al., 1989, “Molecular Cloning, A Laboratory Manual”, 2nd edn., Cold Spring Harbor Laboratory, Cold Spring Harbor, N.Y.). Furthermore, taking the sequence data as a basis, peptides can be synthesized which are employed for obtaining antisera. Gene expression libraries can then be screened using these antisera. The clones resulting from these different screening methods can then be employed, by isolating and sequencing the inserted DNA fragments, for identifying DNA sequence segments which encode the N-terminally sequenced protein segments of the proteins. If the inserted DNA fragments do not contain the complete gene encoding any particular protein, these DNA fragments can be used to isolate the complete genes by screening other gene libraries. The genes which have been completely isolated in this manner can then be expressed, in accordance with the state of the art, in various well-known systems in order to obtain the corresponding protein.

Using oligonucleotides deduced from the N— terminal sequences of SEQ ID NOS: 5, 7, 8, 10, 12 and 15, the genes corresponding to the SEQ ID NOS: 5, 8, 10, 12 and 15 were isolated and are specified as SEQ ID NOS: 20 (catalase), 24 (50 kD membrane protein), 25 (42 kD protein), 26 (36/35/32 kD protein) and 23 (28 kD protein). The gene coding for Hop C could not be isolated using oligonucleotide 7. However, oligonucleotide 7 hybridizes with an homologous gene sp cified as SEQ ID NO: 21 (Hop X). Two additional genes which b long to this family were able to be isolated and are specified as SEQ ID NO: 21 (Hop Y) and SEQ ID NO: 22 (Hop Z).

Another approach is given by the recent access to the complete genomic sequence of H. pylori on the internet which allowed, for example, the identification of SEQ ID NO: 27.

The novel proteins, peptides, antibodies and polynucleotides, and their expression products, can now be used, in accordance with methods known to the skilled person, for preparing a pharmaceutical composition, in particular a vaccine, or a diagnostic agent.

Those regions of the proteins which, on the one hand, occur, if possible, in all H. pylori strains, and, on the other hand, bring about the formation of protective antibodies, are particularly suitable for preparing vaccines. A special preference is given to the regions which project from the surface of the bacteria.

Such vaccines may either be prophylactic (to prevent infection) or therapeutic (to treat disease after infection). These vaccines comprise antigen or antigens, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) those formulations described in PCT Publ. No. WO 90/14837, including but not limited to MF59 (containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.)), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi. Immunochem, Hamilton, Mont.) containing 2% Squalene, 0.2% Tween 80, and one or more bacterial cell wall components from the group consisting of monophosphorylipid A (MPL), trehalose dimycolate (TDt), and cell wall skeleton (CWS), preferably MPL+CWS (Detox™); (3) saponin adjuvants, such as Stimulon™ (Cambridge Bioscience, Worcester, Mass.) may be used or particles generated therefrom such as ISCOMs (immunostimulating complexes); (4) Complete Freunds Adjuvant (CFA) and Incomplete Freunds Adjuvant (IFA); (5) cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumour necrosis factor (TNF), etc; and (6) other substances that act as immunostimulating agents to enhance the effectiveness of the composition. Alum and MF59 are preferred.

As mentioned above, muramyl peptides include, but are not limited to, N-acetyl-muramyl-L-threonyl-D-isoglutamine (thr-MDP), N-acetyl-normuramyl-1-alanyl-d-isoglutamine (nor-MDP), N-acetylmuramyl-1-alanyl-d-isoglutaminyl-1-alanine-2-(1′-2′-dipalmitoyl-sn-glycero-3-huydroxyphosphoryloxy)-ethylamine (MTP-PE), etc.

The immunogenic compositions (e.g., the antigen, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g., nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad rang that can be determined through routine trials.

The immunogenic compositions are conventionally administered parenterally, e.g., by injection, either subcutaneously or intramuscularly. Additional formulations suitable for other modes of administration include oral and pulmonary formulations, suppositories and transdermal applications. Dosage treatment may be a single dose schedule or a multiple dose schedule. The vaccine may be administered in conjunction with other immunoregulatory agents.

The present invention describes, therefore, pharmaceutical compositions, in particular vaccines, and diagnostic agents which comprise one or more of the novel proteins and/or one or more of the novel peptides or one or more of the novel antibodies or one or more of the novel polynucleotides or one or more expression products of the novel polynucleotides.

For example, according to the present invention, a DNA vaccine can be prepared on the basis of the polynucleotides, or a diagnostic agent can be prepared on the basis of the polymerase chain reaction (PCR diagnosis), or an immunotest, for example a Western blot test or an enzyme immunotest (ELISA) can be prepared on the basis of the antibodies. Furthermore, the novel proteins or peptides, or their immunogenic moieties, in particular when they contain an uninterrupted sequence of unambiguously determined amino acids, having a minimum length of five amino acids, preferably six amino acids and, in particular, in the case of the novel peptides having the SEQ ID NOS: 1, 2, 3, 6, 10, 11, 12, 14, 15, 16 and 19 and peptides or proteins encoded by the DNA sequences of SEQ ID NOS: 20, 21, 22, 23, 24, 25, 26 and 27, at least ten consecutive amino acids, can be used as antigens for immunizing mammals. In this context, the two C-terminal regions C1 and C2 specific for H. pylori catalas (c.f. Example 6) can also be used as immunogens. The antibodies which are formed by th immunization, or antibodies which are prepared by means of recombinant DNA methods (see, for example, Winter G. & Milstein C. (1991) Nature, 293-299, Vol. 349), can, inter alia, prevent adhesion of the bacteria to the mucosal surface, attract macrophages for the purpose of eliminating bacteria, and activate the complement system for the purpose of lysing the bacteria.

The following examples are intended to clarify the invention.

### EXAMPLES

### Example 1

### Culture of Helicobacter pylori

The H. pylori stain ATCC 43504 was passaged under microaerophilic conditions (BBL Jar/Campy Pak Plus, from Becton & Dickinson) on Columbia Agar plates containing 5% horse blood (incubation 48 h, 37° C.). Three plates were rinsed off when inoculating a 500 ml flow-spoiler flask (100 ml of Columbia broth, 7% FCS); during the incubation (BBL Jar/Campy Pak Plus; 48 h, 37° C., 90 rpm), the OD₅₉₀ rose from 0.3 to 2.0. The bacteria were harvested by centrifugation at 10,000 rpm and washed twice with physiological sodium chloride solution.

### Example 2

### Isolation of Helicobacter pylori Outer Membrane Proteins

The preparation of the outer membrane protein fraction, with the inner and outer membrane proteins being separated by means of differential solubilization with Sarkosyle (Ciba-Geigy AG), was carried out using the method of Blaser et al. In this method, the bacterial cultures are harvested in the phase of late logarithmic growth, washed in 10 mM Tris buffer (pH 7.4) and disrupted with glass beads in a homogenizer (Institut fur Molekularbiologie und Analytik (IMA), Germany) at 4° C. and 4000 rpm for 15 min. After that, the glass beads are removed by filtration and the bacterial suspension is centrifuged at 5000 g for 20 min in order to remove intact cells. The cell walls are pelleted out of the supernatant by centrifuging at 100,000 g for 60 minutes and at 4° C. The resulting pellet is resuspended with a 1% solution of Sarkosyle in 7 mM EDTA, and the suspension is incubated at 37° C. for 20 min. The Sarkosyle-insoluble fraction, which contains the integral membrane proteins, is pelleted by centrifugation at 50,000 g for 60 minutes and at 4° C. and the pellet is resuspended in sterile distilled water; the suspension is then stored at −20° C.

### Example 3

### SDS Polyacrylamide Gel Electrophoresis and Blotting

Gel preparation, and the electrophoresis, were carried out in a BioRad (Munich) Protean II xi slab cell apparatus. The chemicals employed, and the polyacrylamide monomer (as a 30% solution containing 0.8% bisacrylamide), were obtained from Oxford GlycoSystems (Oxford, UK). In addition to a 10% standard gel, gels containing polyacrylamide contents of 8% and 16% were also especially employed for carrying out separations in the high-molecular weight and low-molecular weight ranges, respectively. The thickness of the gel was 1 mm.

In order to eliminate undesirable oxidizing properties of the ammonium persulphate used for preparing the gel, all the wells of the gel were filled with a solution containing 50 pM of L-methionine/microlitre and left to stand overnight. After the solution has been sucked off on the following day, and after each of the wells has once again been filled with 10 microlitres of this solution in each case, a preliminary electrophoresis takes place. This preliminary treatment prevents the methionine residues of the protein from being oxidized and thereby enables a protein cleavage with BrCN (Met cleavage site) to be carried out if required. The membrane protein fraction starting material is dissolved in 1.5% SDS, 2.5% mercaptoethanol, 5% glycerol and bromophenol blue in 63 mmol/l Tris buffer, pH 6.8, and fractionated by SDS polyacrylamide gel electrophoresis.

Protein transfer from the SDS gel to the PVDF membrane (Immobilon Pe, from Millipore) is carried out in a BioRad (Munich) Trans Blot SD apparatus, under modified conditions.

For the purposes of completing the protein transfer, 0.005% SDS is added to the cathode buffer, thereby counteracting too rapid an impoverishment of SDS in the gel. The use of six filter papers, which are soaked with this buffer, on the cathode side is found to give optimum results in this connection.

The blot was then stained with amidoblack using the protocol of R. Westermeier (Elektrophorese Praktikum (Electrophoresis Laboratory Manual) VCH Verlag Weinheim, 1990, ISBN 3-527-28172-X).

### Example 4

### N-terminal Edman Degradation

The Edman amino acid degradation, and the determination of the PTH amino acids, were carried out in a 477 A liquid phase sequencer having an on-line 120A HPLC analyser (ABI).

For the analyses, the corresponding bands from, in each case, four tracks were cut out of the PVDF blot membrane and sequenced after a washing step, as recommended by ABI.

The number of sequencing steps was 5 to 25 (depending on the quantity of substance available for sequencing).

The Cys and Trp PTH amino acids cannot be detected under the conditions which were chosen.

### Example 5

Deduction felig nucleotides for screening gene libraries and for identifying DNA fragments via Southern Blot analysis.

The following oligonucleotides were deduced from the resulting N-terminal sequences of SEQ ID NOS: 5, 7, 8, 10, 12 and 15:

| | | |
|---|---|---|
| SEQ ID | Oligo- | Amino acid sequence |
| NO: | nucleotide | and predicted nucleotide |

| | | |
|---|---|---|
| 5 | 1 | Val Asn Lys Asp Val Lys Gln Thr Xaa |
| | | GTI AAT AAA GAT GTI AAA CAA ACT TGT |
| | | C C |
| | | Ala Phe Gly Ala Pro |
| | | GCI TTT GGC GCI CCT |
| 7 | 2 | Gly Gly Phe Phe Thr Val Gly Tyr Gln Leu |
| | | GGC GGC TTT TTT ACT GTG GGC TAT CAA TTA |
| | | C G |
| | | Gly Gln Val Met Gln |
| | | GGC CAA GTG ATG CAA |
| 8 | 3 | (Val) (Thr) Tyr Glu Val His (Gly) Asp Phe Ile |
| | | GTG ACT TAT GAA GTG CAT GGC GAT TTT ATC |
| | | C T |
| | | Asn Phe (Ser) Lys Val |
| | | AAT TTT AGC AAA GT |
| | | C |
| 10 | 4 | Lys Glu Lys Phe Asn Arg Thr Lys Pro |
| | | AAA GAA AAA TTT AAC AGA ACC AAA CCT |
| | | T |
| 12 | 5 | Glu Lys Asn Gly Ala Phe Val Gly Ile Ser |
| | | GAA AAA AAT GGI GCI TTT GTG GGC ATT AGC |
| | | C |
| | | Leu Glu Val Gly Arg Ala Asp Gln Lys |
| | | TTI GAG GTT GGI AGA GCT GAT CAA AAA |
| 15 | 6 | Trp Ser Ala Ala Phe Val Gly Val Asn |
| | | TGG AGC GCT GCT TTT GTG GGC GTG AAT |
| | | Tyr Gln Val Ser Met Ile Gln Asn Gln Thr |
| | | TAT CAA GTG AGC ATG ATT CAA AAT CAA ACT |
| | | C C |
| | | Lys Met Val Asn Asp |
| | | AAA ATG GTG AAT GAT |

The oligonucleotides were deduced using the species-specific codon usage of Helicobacter pylori, which had been determined from 19 known H. pylori genes, and using the base inosine (I), which is capable of undergoing stable base pairing with the bases adenine (A), cytosine (C) and thymine (T) with, in each case, two hydrogen bridges. When carrying out the deduction, the degeneracy of the codon was kept as low as possible.

### Example 6

### Isolation and Characterization of the Genes Using the Oligonucleotides Deduced from the Peptide Sequences of SEQ ID NOS: 5, 7, 8, 10, 12 and 15

The oligonucleotides which had been deduced from the peptide sequences of SEQ ID NOS: 5, 7, 8, 10, 12 and 15 were labelled with digoxigenin (DIG) using a kit manufactured by Boehringer Mannheim (DIG Oligonucleotide 3′-End Labelling Kit) and employed for screening a H. pylori gene library which had been prepared using a kit manufactured by Stratagene (Predigested ZAP Express™ BamHI/CIAP Vector Cloning Kit) at 32° C. under standard conditions. Using oligonucleotides 1, 3 and 6, it was possible to identify clones which carry DNA fragments containing sequences which encode the peptide sequences of SEQ ID NOS: 5, 8 and 15. Oligonucleotide 2 hybridized with a DNA fragment which encodes an homologous sequence of SEQ ID NO: 7.

Using oligonucleotides 4 and 5, it was only possible to isolate clones whose DNA fragments did not encode SEQ ID NOS: 10 and 12. This is why these oligonucleotides and the clones which had been isolated from the λZAP Express gene library were employed in a Southern Blot analysis, which permitted the unequivocal identification of DNA fragments which hybridized with the oligonucleotides, but not with the DNA fragments resulting from the screening. With these DNA fragments, in each case one sub-gene library was prepared in the λZAP Express vector, and each sub-gene library was screened with oligonucleotides 4 and 5. This allowed the identification of clones which carry DNA fragments encoding the sequences of SEQ ID NOS: 10 and 12.

Partial digestion of H. pylori DNA using the restriction enzymes Sau3AI, AluI and HaeIII gave a DNA which was used for establishing gene libraries in the vector λTriplex (Clontech). These gene libraries were used as starting material for isolating the complete genes of the above-described DNA fragments using standard methods.

SEQ ID NO: 20 describes the DNA sequence which encodes the catalase of H. pylori. The nucleotide region 337 to 378 describes the hybridization site with oligo-nucleotide 1. The catalase gene of H. pylori has been described in 1996 by Stefan Odenbreit, Börn Wieland and Rainer Haas (J. Bacteriol. 178, 6960-6967) and is therefore not new. However, when comparing the amino acid sequences of the catalases of Escherichia coli, Bacillus firmus, B. subtilis A, B. subtilis B, rats, mice, cattle, humans, Staphylococcus violaceus, Haemophilus influenzae, B. fragilis, Pseudomonas mirabilis, B. pertussis and P. syringae with the amino acid sequence of H. pylori, it is possible to identify two C-terminal regions C1 (RDPKFNLAHIEKEFEVWNWDYRA) and C2 (EKHQKMMKDMHGKDMHHTKKKK), which are specific to H. pylori catalase. These two peptides were synthesized using standard techniques, coupled to KLH and used for immunizing rabbits. These rabbits developed antibodies against the two peptides, which reacted in the Western Blot analysis with H. pylori catalase which had been produced by recombinant technique. These H. pylori-catalase-specific regions may conceivably be used for developing a vaccine which avoids the problem complex of autoimmune reactions or for the development of a diagnostic which reacts specifically with H. pylori catalase.

SEQ ID NO: 21 describes a nucleotide sequence which was identified by hybridization with the oligo-nucleotide 2. The oligonucleotide hybridized with the sequence of nucleotide 1240 to 1284. This encodes a sequence which is homologous to the porin Hop C (Exner et al., 1995) and is identical with the published amino-terminal sequence EDDGGFFTVGYQLGQVMQDVQNPG in positions 1, 2, 3, 4, 9, 10, 11, 12, 14, 18 and 22.

The porins Hop A, Hop B, Hop C and Hop D have identical amino acids in 9 positions of the 20 N-terminal amino acids (Exner et al., 1995). In 8 of these positions, there are identical positions also in the sequence described in the present publication; in the 9th position, a conserved amino acid exchange is present (Val-Ile). It can thus be assumed that the protein described in the present publication is equally part of this group of the porins; it was therefore termed Hop X.

On the basis of the homology data and on the basis of the N-terminal sequence determined and on the basis of the hydrophobicity of the N-terminal protein sequence deduced from the nucleic acid sequence, it can be concluded that the protein deduced has a signal sequence. The mature protein with 428 amino acids has a molecular weight of 47.3 kD and an isoelectric point of 10.0.

A further open reading frame was found upstream of the gene which encodes Hop X. This further open reading frame encodes a protein which is homologous to Hop X (34% identity) and which was therefore termed Hop Y. The gene region found to date encodes the 361 C-terminal amino acids of the protein. The gene region as yet outstanding is currently being isolated using standard techniques.

We have thus identified a gene region of H. pylori which encodes at least two porins which are connected in series.

SEQ ID NO: 22 describes a nucleotide sequence which was concomitantly isolated and sequenced during the screening process. The amino acid sequence deduced encodes the 392 C-terminal residues of a protein which shows a high homology with Hop X (33% identity) and Hop Y (28% identity) and which was therefore termed Hop Z. The gene region which encodes the N-terminal portion of the protein is currently being isolated.

SEQ ID NO: 23 describes a DNA sequence which encodes a hitherto undescribed protein. The nucleotide region 696 to 767 describes the hybridization site with the oligonucleotide 6. On the basis of the N-terminal protein sequence which has been determined, in which it was not possible unequivocally to determine the amino acids in the first two positions, and on the basis of the hydrophobicity of the N-terminal protein sequence deduced from the nucleic acid sequence, it can be concluded that the protein deduced has a signal sequence of 17 amino acids. The mature protein of 231 amino acids has a molecular weight of 26.4 kD and an isoelectric point of 10.3. Thus, the molecular weight is quite close to the molecular weight of 28 kD which had been determined by SDS gel electrophoresis. The amino acid sequence deduced is homologous with the sequences of the proteins Hop X, Hop Y and Hop Z, for which the GCG Bestfit Programme determined identity values of 41%, 38% and 41%, respectively. The 28 kD protein thus also seems to be part of the family of the porins or porin-like proteins.

SEQ ID NO: 24 describes a DNA sequence which encodes the non-heat-modifiable 50 kD membrane protein. This protein was first described by Exner et al., 1995, and an N-terminal sequence of the protein was determined. Using the approach described by us, we were then able to describe, with SEQ ID NO: 8, an N-terminal sequence which is identical to the sequence described by Exner et al. (1995), with the aid of the oligonucleotide 3, which had been deduced using the method illustrated in Example 5 and had been used for screening a H. pylori gene library using the above-described methods, it was then possible to identify a DNA fragment which encodes the 50 kD membrane protein. Using other standard methods; it was then possible to determine the nucleic acid sequence described in SEQ ID NO: 24, which encodes a mature protein of 499 amino acids which has a molecular weight of 56.3 kD and an isoelectric point of 9.75. Due to the data of the N-terminal sequencing procedures and the hydrophobicity of the N-terminal sequence, a signal sequence of 29 amino acids is assumed. The amino acid residues 236 to 254 contain a hydrophobic region which is large enough to act as a transmembrane region. Based on such data and using standard methods for epitope analysis, it is possible to identify regions which might be presented on the surface of bacteria. Such regions might be used for developing a vaccine or a diagnostic.

SEQ ID NO: 25 describes a DNA sequence 2825 bp in size which was identified by means of hybridization with oligonucleotide 4, which was deduced from SEQ ID NO: 10. Oligonucleotide 4 hybridized with the nucleotide region 897 to 923 of the described sequence of SEQ ID NO: 25. The protein has no signal sequence. The encoding region of SEQ ID NO: 25 codes for a protein of 399 amino acids with a molecular weight of 43.6 kD and an isoelectric point of 5.0. A search for homologous sequences using the BLASTP program (S. F. Altschul et al., 1990, J. Mol. Biol. 215, 403-410) identified the 42 kD antigen of H. pylori as the elongation factor TU. The maximum percentage of identity (89%) was found with the elongation factor TU from Wolinella succinogenes (W. Ludwig et al., 1993, Antonie van Leeuwenhoek 64, 285-305).

SEQ ID NO: 26 describes a DNA sequence 2182 bp in size which hybridizes with oligonucleotide 5, which had been deduced from SEQ ID NO: 12. Oligonucleotide 5 hybridized with a Sau3AI fragment (position 1 to 575) of the gene library starting from position 524. The screening of different DNA libraries with specific oligonucleotides allowed the isolation of the complete gene described in SEQ ID NO: 26. An amino acid sequence which is identical to the one from SEQ ID NO: 12 can be deduced from SEQ ID NO: 26. Both protein sequencing and the hydrophobicity of the N-terminal sequence deduced allow the conclusion that the antigen has a signal sequence. The mature protein consists of 328 amino acid residues with a molecular weight of 36.1 kD and an isoelectric point of 9.95. No homologous proteins were identified using the BLASTP program (S. F. Altschul et al., 1990).

The sequences described in SEQ ID NOS: 20 to 26 indicate nucleotide sequences which encode antigens of the H. pylori strain ATCC 43504. However, it is known for H. pylori that heterogeneity between identical antigens may exist amongst various strains. We therefore claim not only the sequences described in SEQ ID NOS: 21 to 26, but in addition also the sequences of other H. pylori strains which are homologous with the sequences described herein.

### Example 7

### Identification and Isolation of Genes from H. pylori Corresponding to the Peptide Sequences Listed in Tables 1a-1a Using the Access to the Genomic Sequence

The Institute for Genomic Research (TIGR) released the DNA sequence from H. pylori on 24th Jun. 1997. This new information can be accessed on the internet at “www.tigr.org”. Using the TBLASTN program (Altschul et al., 1997, Nucleic Acids Research 25, in press) the peptide sequences listed in Tables 1a-1c can be aligned to amino acid sequence data deduced from all six reading frames of the H. pylori strain 26695. Having access to the genomic DNA sequence, DNA sequences corresponding to the aligned amino acid sequences can be identified using GCG (Genetic Computer Group) programs. This approach is shown for SEQ ID NO: 19, for example. The sequence of SEQ ID NO: 19 aligned with a very similar sequence using the TBLASTN program. SEQ ID NO: 27 describes the nucleic acid sequence and deduced amino acid sequence from the coding region of a H. pylori gene (strain 26695) localised between position 843212 and 843691 of the genomic sequence. The protein has no signal sequence. The N-terminal sequence of SEQ ID NO: 19 is highly homologous to the N-terminal region of the deduced amino acid sequence from amino acid residue 1 to 15. Only one different amino acid residue is present at position 4: the nucleotide sequence found by the alignment encodes a Ser residue in this position instead of an Asn residue determined by N-terminal sequencing. This can be explained by strain specific differences. The identified nucleic acid sequence in SEQ ID NO: 27 codes for a protein of 159 amino acid residues with a molecular weight of 18.2 kD and an isoelectric point of 7.2. The molecular weight is very close to that of 17 kD determined from SDS polyacrylamide gel electrophoresis. A search for homologous sequences using the BLASTP program (S. F. Altschul et al., 1990) shows that the 17 kD antigen is very homologous to “hydroxymyristol-[acyl carrier protein] dehydratase” from different bacteria.

TABLE 1a
N-terminal sequences of Heliobacter pylori membrane proteins

| | | | | |
|---|---|---|---|---|
| | Molecu- | | | |
| SEQ | lar | | | |
| ID | weight | | | Identi- |
| NO: | (kD) | Sequence | Features | fication |

| | | | | |
|---|---|---|---|---|
| 1 | −250 | Xaa Pro Asn Gly Xaa Tyr Met Xaa Arg Xaa | Xaa at positions 1, 5, 12, 14 and 16 are | unknown |
| | | 5 10 | unknown amino acids. At position 8, Xaa is | |
| | | Xaa Xaa Ile Xaa Xaa Xaa Gln | probably Gln, while at position 10 it is | |
| | | 15 | probably Ser, at position 11 it is probably | |
| | | | Tyr and at position 15 it is probably Thr. | |
| 2 | −110 | Xaa Lys Leu Xaa Pro Gln Xaa Gly Tyr Val | At position 1, Xaa is an unknown amino acid. | unknown |
| | | 5 10 | At position 4, Xaa is Ile or Thr and at | |
| | | Leu Met Tyr | position 7 it is Ala or Lys. | |
| 3 | −100 | Xaa Gln Asp Xaa Phe Leu Xaa Glu Gly Xaa | Xaa at positions 1 and 10 are unknown amino | unknown |
| | | 5 10 | acids, and at position 4, Xaa is Ile or Thr | |
| | | Ser | and at position 7 it is Ala or Lys. | |
| 4 | 62 | Xaa Lys Lys Ile Ser Arg Lys Glu Tyr Val | At position 1, Xaa is probably Met. | urease B |
| | | 5 10 | | |
| | | Ser Met Tyr Gly Pro | | |
| | | 15 | | |
| 5 | 60 | Xaa Val Asn Lys Asp Val Lys Gln Thr Xaa | Xaa at positions 1 and 10 are unknown amino | 63 kD |
| | | 5 10 | acids. | exoenzym |
| | | Ala Phe Gly Ala Pro | | e-like |
| | | 15 | | adhesin |
| 6 | 60 | Xaa Phe Gln Val Xaa Phe Xaa Ile Xaa Ala | Xaa at positions 1, 5 and 9 are unknown | unknown |
| | | 5 10 | amino acids, and at position 7 Xaa is Ala or | |
| | | Met Asn | Leu. | |
| 7 | 50 | Xaa Xaa Xaa Gly Gly Phe Phe Thr Val Gly | At positions 2, 3 and 19, Xaa are unknown | Hop C |
| | | 5 10 | amino acids, and at position 1 Xaa is | |
| | | Tyr Gln Leu Gly Gln Val Met Gln Xaa Val | probably Glu. | |
| | | 15 20 | | |

TABLE 1b

| | | | | |
|---|---|---|---|---|
| | Molecu- | | | |
| SEQ | lar | | | |
| ID | weight | | | Identi- |
| NO: | (kD) | Sequence | Features | fication |

| | | | | |
|---|---|---|---|---|
| 8 | 50 | Xaa Xaa Tyr Glu Val His Xaa Xaa Xaa Ile | Xaa at positions 1, 2, 7 and 13 are | 50 kD |
| | | 5 10 | unknown, and at position 8 Xaa is pro- | membrane |
| | | Asn Phe Xaa Lys Val | bably Asp and at position 9 it is pro- | protein |
| | | 15 | bably Phe. | |
| 9 | 49 | Xaa Xaa Asp Gly Xaa Phe Met Thr Phe Gly | Xaa at positions 1, 2 and 5 are unknown | Hop B |
| | | 5 10 | | |
| | | Tyr Glu Leu Gly Gln | | |
| | | 15 | | |
| 10 | 42 | Xaa Lys Glu Lys Phe Xaa Arg Thr Lys Pro | Xaa at positions 1 and 11 are unknown, | unknown |
| | | 5 10 | while at position 6, Xaa is probably | |
| | | Xaa Val Xaa Xaa | Asn or Gln, at position 13 it is | |
| | | | probably Thr and at position 14 it is | |
| | | | probably Ile. | |
| 11 | 42 | Xaa Gly His Xaa Gln Xaa His Xaa Ala Gln | Xaa at positions 1 and 4 are unknown, | unknown |
| | | 5 10 | while at position 6 Xaa is Asn or Gln | |
| | | | and at position 8 it is probably Pro. | |
| 12 | 36/35/32 | Xaa Glu Lys Asn Gly Ala Phe Val Gly Ile | Xaa at position 1 is unknown, while at | unknown |
| | | 5 10 | position 21 it is probably Thr. | |
| | | Ser Leu Glu Val Gly Arg Ala Asp Gln Lys Xaa | | |
| | | 15 20 | | |
| 13 | 31 | Met Lys Leu Thr Pro Lys Glu Leu Asp Lys | ----------- | urease A |
| | | 5 10 | | |
| | | Leu Met Leu His Tyr Ala Gly Glu Leu Ala | | |
| | | 15 20 | | |

TABLE 1c

| | | | | |
|---|---|---|---|---|
| | Molecu- | | | |
| SEQ | lar | | | |
| ID | weight | | | Identi- |
| NO: | (kD) | Sequence | Features | fication |

| | | | | |
|---|---|---|---|---|
| 14 | 30 | Xaa Glu Phe Ala Gln Phe Val Gly Val Asn | Xaa at positions 1 and 13 are unknown | unknown |
| | | 5 10 | amino acids. | |
| | | Tyr Gln Xaa Asn | | |
| 15 | 28 | Xaa Xaa Ser Ala Ala Phe Val Gly Val Asn | Xaa at position 1 is an unknown amino | unknown |
| | | 5 10 | acid, while at position 2 it is probably | |
| | | Tyr Gln Val Ser Met Ile Gln Asn Gln Thr | Trp. | |
| | | 15 20 | | |
| | | Lys Met Val Asn Asp | | |
| | | 25 | | |
| 16 | 28 | Xaa Xaa Xaa Ile Xaa Xaa Xaa Leu Tyr Xaa | Xaa at positions 1, 2, 3, 6, 10 and 14 | unknown |
| | | 5 10 | are unknown amino acids, while at | |
| | | Leu Met Leu Xaa Arg | position 5, Xaa is Pro or Val and at | |
| | | 15 | position 7 it is probably Lys. | |
| 17 | 25 | Xaa Gln Arg Met Xaa Gln Val Gly | Xaa at position 1 is an unknown amino | unknown |
| | | 5 | acid, while at position 5 Xaa is Pro or | |
| | | | Lys. | |
| 18 | 25 | Xaa Leu Asn Ile Xaa Phe Ala | Xaa at position 1 is an unknown amino | unknown |
| | | 5 | acid, while at position 5 Xaa is Pro or | |
| | | | Lys. | |
| 19 | 17 | Xaa Glu Gln Asn Xaa Gln Asn Leu Gln Xaa | Xaa at positions 1, 5 and 10 are unknown | unknown |
| | | 5 10 | amino acids, while at position 11 Xaa is | |
| | | Xaa Phe Phe Ile Xaa | probably Gln and at position 15 it is | |
| | | 15 | probably Lys. | |

1-34. (Canceled)

35. An isolated Helicobacter pylori-specific peptide comprising a minimum sequence of eight consecutive amino acids deduced from SEQ ID NO:36, wherein said sequence comprises a T-cell or B-cell epitope of Helicobacter pylori.

36. An isolated Helicobacter pylori-specific polynucleotide comprising a nucleic acid sequence encoding the peptide of claim 35.

37. An expression vector comprising the polynucleotide of claim 36 operably linked to regulatory regions that facilitate expression of said polynucleotide.

38. A host cell transformed with the polynucleotide of claim 36.

39. A host cell comprising the expression vector of claim 37.

40. The peptide of claim 35, wherein said peptide is recombinantly expressed.

41. A pharmaceutical composition comprising the peptide according to claim 35 and a pharmaceutically acceptable carrier.

42. A pharmaceutical composition, which comprises the isolated polynucleotide according to claim 36 and a pharmaceutically acceptable carrier.

43. A diagnostic agent comprising the peptide according to claim 35.

44. An isolated Helicobacter pylori specific peptide comprising a minimum sequence of eight consecutive amino acids from SEQ ID NO:37, wherein said sequence comprises a T-cell or B-cell epitope of Helicobacter pylori.

45. The peptide of claim 35 wherein the minimum sequence is ten consecutive amino acids deduced from SEQ ID NO:36.

46. The pharmaceutical composition of claim 41, which additionally comprises an adjuvant.

47. A pharmaceutical composition comprising the isolated polynucleotide of claim 36 and a pharmaceutically acceptable carrier.

48. A pharmaceutical composition comprising the peptide of claim 40 and a pharmaceutically acceptable carrier.

49. The pharmaceutical composition of claim 42, which additionally comprises an adjuvant.

50. The pharmaceutical composition of claim 47, which additionally comprises an adjuvant.

51. A diagnostic agent comprising the peptide according to claim 40.

52. A diagnostic agent comprising the polynucleotide of claim 36.

53. The pharmaceutical composition of claim 42, wherein said polynucleotide is operably linked to regulatory regions that facilitate expression of said isolated nucleic acid.

54. The pharmaceutical composition of claim 47, wherein said polynucleotide is operably linked to regulatory regions that facilitate expression of said isolated nucleic acid.

55. A polynucleotide comprising an isolated nucleic acid sequence encoding the peptide of claim 44.

56. A pharmaceutical composition comprising the peptide according to claim 44 and a pharmaceutically acceptable carrier.

57. A diagnostic agent comprising a peptide according to claim 44.

58. The peptide of claim 44 wherein the minimum sequence is ten consecutive amino acids from SEQ ID NO:37.

59. The peptide of claim 44, wherein said peptide is recombinantly expressed.
